(19) United States
(12) Patent Application Publication
(10) Pub. No.: US 2005/0075388 A1
(43) Pub. Date: Apr. 7, 2005

(54) Products of condensations of hydroxycoumarin derivatives with aromatic and aliphatic dialdehydes, their preparation and antiviral action thereof

(76) Inventors: Zrinka Ivezic, Zagreb (HR); Mladen Trkovnik, Zagreb (HR)

Correspondence Address: CONNOLLY BOVE LODGE & HUTZ LLP, SUITE 800, 1990 M STREET NW, WASHINGTON, DC, 20036-3425, US

(21) Appl. No.: 10/491,369

(22) PCT Filed: Oct. 1, 2001
(86) PCT No.: PCT/HR01/00044

(51) Int. Cl. A61K 31/366; C07D 45/14

(52) U.S. Cl. 514/457; 514/432; 549/23; 549/280; 549/284

(57) ABSTRACT

Disclosed are a novel class of components obtained in reactions of condensation of hydroxycoumarin derivatives with aromatic and aliphatic dialdehydes, their preparation and antiviral action thereof, such as against HIV.

### TECHNICAL FIELD

IPC: C07D 311/04

The present invention relates to novel derivatives of mono, di and trihydroxycoumarins condensed with aromatic and aliphatic dialdehydes. Objects of the invention are also processes for preparing these hydroxycoumarin derivatives condensed with aromatic and aliphatic dialdehydes as well as antiviral action of these compounds. Preliminary investigations have shown that some compounds of the present invention exhibit an anti-HIV action.

### BACKGROUND OF THE INVENTION

Hydroxycoumarin derivatives showing an antiviral action against human immunodeficiency virus are well-known (H. I. Sckulnick et al., J. Med. Chem. 40 (1997) 1149). This discovery has led to an enhanced interest for compounds of hydroxycoumarin class and has quickly resulted in numerous works wherein novel hydroxycoumarin derivatives and the anti-HIV action thereof have been investigated. There are disclosed numerous products of condensation of hydroxycoumarins with aromatic and aliphatic monoaldehydes, wherein the importance of the existence of more than one hydroxyl group in a coumarin unit in order to improve the virostatic action has been emphasized (H. Zhao et al. J, Med. Chewn. 40 (1997) 242). The phenomenon of resistance to known HIV inhibitors necessitates the identification of novel compounds having an improved antiviral action.

Hydroxy- and polyhydroxycoumarin derivatives formed by condensation with aldehydes and aldehyde carboxylic acids and having an anti-HIV action are disclosed in U.S. Pat. No. 6,100,409. According to our knowledge other condensation products between hydroxycoumarins and aliphatic dialdehydes have not been disclosed in the literature.

The present invention discloses a novel class of compounds formed in condensation reactions of hydroxycoumarin derivatives with aromatic and aliphatic dialdehydes, their preparation and the antiviral action thereof.

The object of the invention are products of condensation of hydroxycoumarin derivatives with aromatic and aliphatic dialdehydes of the general formulae I, II, III, IV, and V: 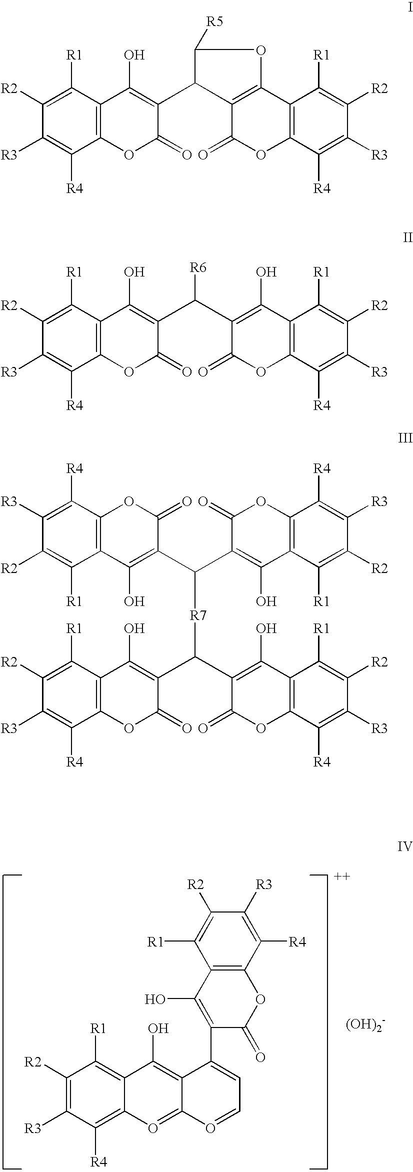 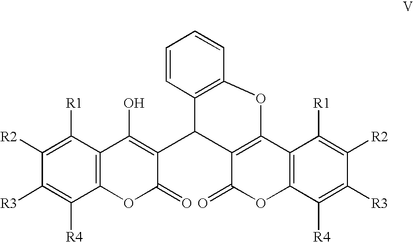
wherein
is a single or a double bond;
R1=R2=R3=R4=H or
R1=R2=R4=H, R3=OH or
R2=R4=H, R1=R3=OH or
R1=R4=H, R2=R3=OH or
R1=R2=H, R3=R4=OH;
R5=H, OCH₃ or OCH₂CH₃;
R6=o-C₆H₄—CHO, CHO;
R7=(CH₂), and n=1-3,
and pharmaceutically acceptable salts and esters thereof.

One of the objects of the present invention are also processes for the preparation of novel compounds of the general formulae I-V with R groups defined as stated. According to the present invention novel derivatives of mono, di and trihydroxy-coumarins condensed with aromatic and aliphatic dialdehydes are prepared starting from hydroxycoumarins of the general formula VI 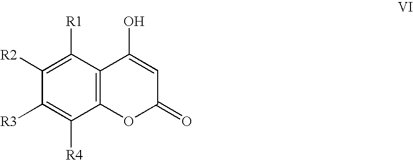
wherein R1-R4 have the above meanings, in reactions of condensation with aliphatic and aromatic dialdehydes of the general formula VII 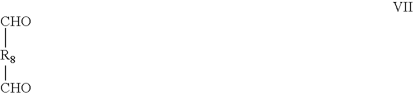
wherein
R8=ortho-phenyl or (CH₂)ₙ and n=0-3.

Novel hydroxycoumarin derivatives of the present invention exhibit an antiviral action against HIV.

The term “pharmaceutically acceptable salts” as used relates to those salts which, according to known medical estimations, are suitable for use in contact with human tissues and tissues of higher animals and will not cause toxicity, irritations, allergies etc. Pharmaceutically acceptable salts are well-known, e.g. S. M. Berge et al. in J. Pharm. Sci. 66 (1977) 1 disclose pharmaceutically acceptable salts in detail. These salts may be prepared in situ during final isolation and purification of the present compounds or separately in the reaction with an appropriate organic acid or base. Examples of pharmaceutically acceptable non-toxic salts are salts of amino group formed by the reaction with inorganic acids such as hydrochloric, hydrobromic, phosphoric, sulfuric and perchloric acids, or with organic acids such as acetic, oxalic, maleic, tartaric, citric, succinic or malonic acids. Other pharmaceutically acceptable salts include alginates, ascorbates, aspartates, benzenesulfonates, benzoates, bisulfates, borates, butyrates, camphorates, camphorsulfonates, citrates, cyclopentanepropionates, digluconates, dodecylsulfates, ethanesulfonates, formiates, phosphates, fumarates, glucoheptonates, glycerophosphates, gluconates, hemisulfates, heptanoates, hexanoates, 2-hydroxyethanesulfonates, lactobionates, lactates, laureates, lauryl sulfates, malates, maleates, malonates, methanesulfonates, 2-naphthalenesulfonates, nicotinates, nitrates, oleates, oxalates, palmitates, persulfates, 3-phenylpropionates, picrates, pivalates, propionates, stearates, succinates, sulfates, tartrates, thiocyanates, p-toluenesulfonates, undecanoates, valerates etc. Representative alkali metal salts and earth alkali metal salts include sodium, lithium, potassium, calcium, magnesium and similar salts. Further, pharmaceutically acceptable salts include non-toxic ammonium salts, quarternary ammonium salts and amine cations formed by formation of counterions such as halides, hydroxides, carboxylates, sulfates, phosphates, nitrates, lower alkyl and aryl sulfonates.

The term “pharmaceutically acceptable esters” relates to esters which hydrolyze in vivo and include those esters which are regularly broken in human organism to leave only the starting substance or its salt. Suitable ester groups include e.g. those derived from pharmaceutically acceptable aliphatic carboxylic acids, especially alkanoic, alkenoic and cycloalkanoic acids, wherein each alkyl or alkenyl unit has no more than six carbon atoms. Examples of such esters include formates, acetates, propionates, butyrates, acetylates and ethylsuccinates.

### PREPARATION OF COMPOUNDS OF THE PRESENT INVENTION

Novel compounds of the present invention are prepared in reactions of hydroxycoumarins of the formula VI, wherein R1-R4 have above meanings, with aliphatic or aromatic dialdehydes of the general formula VII with the above meanings of the R group. Reactions are presented in schemes 1-4 wherein the substituents have the above meanings if not expressly stated otherwise.

Scheme 1 illustrates the condensation of hydroxycoumarins of the general formula VI with the dialdehyde of the general formula VII wherein R8 has the meaning of (CH₂)ₙ and n=0 (glyoxal) in methanol or ethanol at a temperature from room temperature to the temperature of the boiling point of the solvent to obtain compounds of the general formula I wherein—has the meaning of a single bond and R5=OCH₃ or OCH₂CH₃ and—has the meaning of a double bond and R5=H.

When the condensation with said reagents is carried out at room temperature, besides said compounds also the compound of the general formula II wherein R6 has the meaning of CHO is isolated.

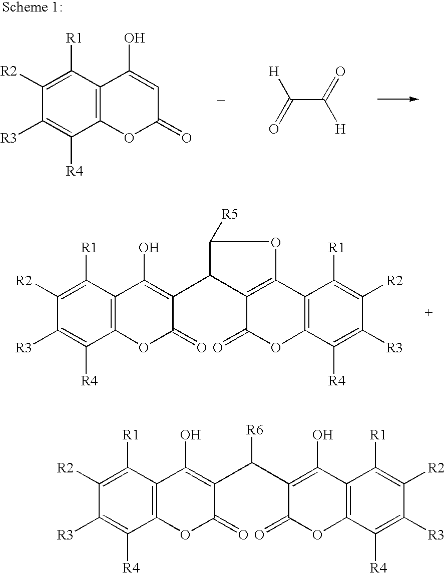

Scheme 2 illustrates a process for preparing compounds of the general formula III wherein R7 has the meaning of (CH₂)ₙ and n=1, and of the general formula IV. The condensation is carried out by reaction of hydroxycoumarins of the general formula VI and of dialdehydes of the general formula VII wherein R8 has the meaning of (CH₂)ₙ and n=1 (malonaldehyde), in ethanol at boiling temperature for 9 to 33 hours.

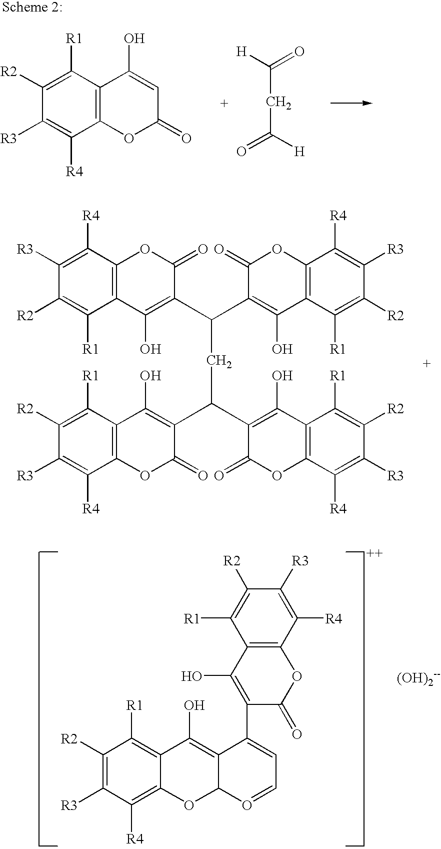

Scheme 3 ilustrates the preparation of the compounds of the general formula III wherein R7 has the meaning of (CH₂)ₙ and n=3, by reaction of condensation of hydroxycoumarins of the general formula VI with dialdehyde of the general formula VII wherein R8=(CH₂)ₙ and n=3 (glutardialdehyde) in ethanol at the temperature of the boiling point of the solvent for 8 to 16 hours.

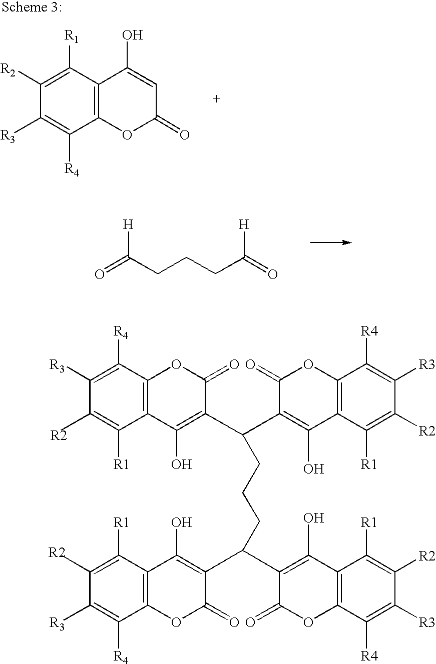

Scheme 4 illustrates the preparation of the compounds of the general formula II wherein R5 has the meaning of o-C₆H₄—CHO, and of the compounds of the general formula V by the reaction of condensation of hydroxycoumarins of the general formula VI with phthalaldehyde (benzene-1,2-dicarbaldehyde) in a suitable organic solvent, preferably acetone or methanol, for 38 to 109 hours at the temperature of the boiling point of the solvent.

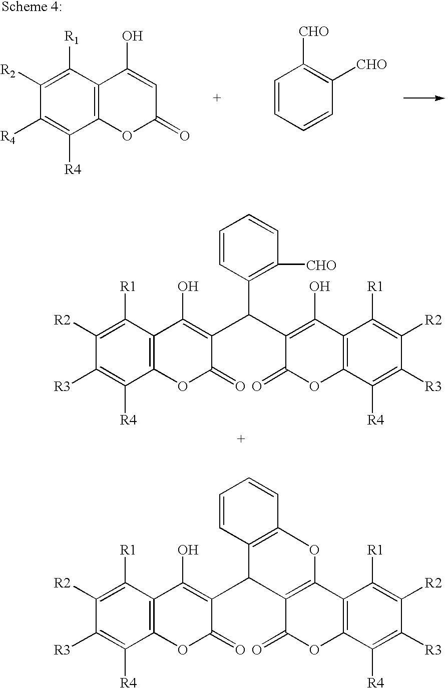

Preliminary investigations have shown that some of the compounds according to the present invention exhibit an anti-HIV action.

The present invention is illustrated by the following Examples which in no way should be construed as limitative for the invention.

### EXAMPLE 1

### 2-Ethoxy-3-(4-hydroxy-2-oxo-2H-chromen-3-yl)-2,3-dihydro-furo[3,2-c]chromen-4-one

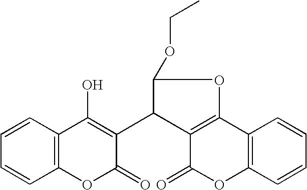

A 30% aqueous glyoxal solution (0.28 ml; 1.70 mmol) was added to a solution of 4-hydroxycoumarin (1.00 g; 6.17 mmol) in ethanol (10.0 ml) and heated at the temperature of the boiling point of ethanol for nine hours. The reaction mixture was filtered off and the precipitate was recrystallized from glacial acetic acid. 3,3′,3″,3′″-(1,2-ethane)tetrakis[4-hydroxycoumarin] (0.61 g; 59%) was obtained. To the filtrate wherefrom the precipitated 3,3′,3″,3′″-(1,2-ethane)tetrakis[4-hydroxycoumarin] was separated, another 0.1 ml of glyxal was added and after six hours a white precipitate was separated, which was washed several times with hot 96% ethanol. The obtained 2-ethoxy-3-(4-hydroxy-2-oxo-2H-chromen-3-yl)-2,3-dihydro-furo[3,2-c]chromen-4-one had a m.p. of 166-169° C.

Elemental analysis: C₂₂H₁₆O₇ (Mᵣ=392.36) Calc.: C=65.75% H=5.06%. Found: C=65.64% H=5.04%. MS n/z: FAB: 393 (MH⁺), 347. IR (KBr): v/cm⁻¹: 3390; 1722; 1609; 1565; 1237; 761. ¹H-NMR (600 MHz, DMSO-d₆): δ/ppm: 12.90 (bs, 1H, C4-OH); 8.02 (d, J=8.2 Hz, 1H, H5); 7.80 (d, J=7.8 Hz, 1H, H5′); 7.71 (t, J₁=7.9 Hz, J₂=7.9 Hz, 1H, H7′); 7.65 (t, J₁=7.5 Hz, J₂=7.5 Hz, 1H, H7); 7.47 (d, J=8.3 Hz, 1H, H6′); 7.43 (t, J₁=7.6 Hz, J₂=7.6 Hz, 1H, H8′); 7.39-7.38 (m, 2H, H6 and H8); 6.20 (s, 1H, H5-furan); 4.87 (s, 1H, H4-furan); 3.99 (q, 2H, OCH₂CH₃); 3.44 (q, 2H, CH₃CH₂OH); 1.21 (t, 3H, OCH₂CH₃); 1.08 (t, 3H, CH₃CH₂OH). ¹³C-NMR (600 MHz, DMSO-d₆): δ/ppm: 165.02 (C4′-OH); 162.03 (C4); 161.00 (C2′); 158.23 (C2); 154.53 (C9′); 152.19 (C9); 132.94 (C7′), 132.52 (C7); 124.42 (C6′); 124.08 (C6); 123.58 (C5′); 122.71 (C5); 116.71 (C8′); 116.36 (C8); 115.92 (C10); 113.72 (C5-furan); 112.01 (C10′); 102.07 (C3′); 101.00 (C3); 65.13 (OCH₂CH₃); 55.98 (CH₃CH₂OH); 42.81 (C4-furan); 18.52 (CH₃CH₂OH); 15.04 (OCH₂CH₃).

### EXAMPLE 2

### 3-(4,7-Dihydroxy-2-oxo-2H-chromen-3-yl)-7-hydroxy-furo[3,2-c]chromen-4-one

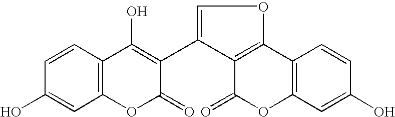

A 30% aqueous glyoxal solution (0.13 ml; 0.77 mmol) was added to a solution of 4,7-dihydroxycoumarin (0.50 g; 2.81 mmol) in ethanol (5.0 ml) and heated at the temperature of the boiling point for 11 hours with a yellow precipitate separating from an orange solution. The reaction mixture was left overnight at 13° C. and filtered. The light yellow precipitate was recrystallized from glacial acetic acid. A light yellow precipitate of 3-(4,7-dihydroxy-2-oxo-2H-chromen-3-yl)-7-hydroxy-furo[3,2-c]chromen-4-one (1.20 g; 47%) having a m.p.>300° C. was obtained.

Elemental analysis: C₂₀H₁₀O₈ (Mᵣ=378.280) Calc. : C=63.50% H=2.66%. Found: C=62.93% H=3.65%. MS mn/z EI: 378 (M⁺) IR (KBr): v/cm⁻¹: 3388; 1686; 1628; 1587; 1476; 1296; 815; 770. ¹H-NMR (300 MHz, DMSO-d₆): δ/ppm: 12.34 (bs, 1H, C4-OH); 10.78-10.59 (bs, 2H, C7-OH); 7.88 (d, J=8.7 Hz, 1H, H5); 7.77 (d, J=8.4 Hz, 1H, H5′); 7.12 (s, 1H, CH); 6.92-6.83 (m, 2H, H6, H6′); 6.74 (s, 2H, H8, H8′). ¹³C-NMR (300 MHz, DMSO-d₆): δ/ppm: 164.33 (C4); 162.82 (C2); 160.92 (C4′), 160.73 (C2′); 157.93 (C7′); 157.45 (C7); 154.84 (C9′); 154.16 (C9); 148.58 (CH-furan); 125.96 (C7′); 122.53 (C7); 113.77 (C3-furan); 113.43 (C3′); 108.60 (C10′); 108.29 (C10); 107.91 (C6′); 104.65 (C6); 103.20 (C8′); 102.19 (C8); 92.67 (C3).

### EXAMPLE 3

### 3-(4,7-Dihydroxy-2-oxo-2H-chromen-3-yl)-7-hydroxy-2-methoxy-2,3-dihydro-furo[3,2-c]chromen-4-one

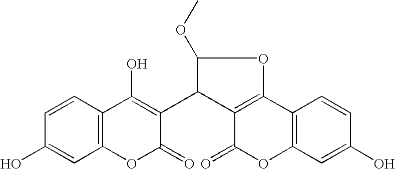

A 30% aqueous glyoxal solution (0.21 ml; 1.24 mmol) was added to a solution of 4,7-dihydroxycoumarin (0.80 g; 4.47 mmol) in methanol (16.0 ml) and stirred at room temperature for 144 hours with a light yellow precipitate separating from an orange solution. The reaction mixture was left overnight at 4° C. and filtered. 1.25 g of a precipitate were obtained, wherefrom 800.0 mg were taken and purified on a silica gel column with the system dichloromethane and methanol (5:1) as the eluant. The fractions were separated and characterized. 3-(4,7-Dihydroxy-2-oxo-2H-chromen-3-yl)-7-hydroxy-2-methoxy-2,3-dihydro-furo[3,2-c]chromen-4-one (90.0 mg; 15%) was obtained. M.p.:>300° C.

Elemental analysis: C₂₁H₁₄O₉ (Mᵣ=410.322) Calc. : C=61.47% H=3.44%. Found: C=61.11% H=3.82%. MS m/z EI: 409 (M−H); 377. IR (KBr): v/cm⁻¹: 3361; 1697; 1666; 1581; 1437; 1293; 765. ¹H-NMR (300 MHz, DMSO-d₆): δ/ppm: 12.30 (bs, 1H, C4-OH), 10.18 (bs, 2H, C7-OH); 7.63 (d, J=8.7 Hz, 1H, H5); 7.54 (d, J=8.4 Hz, 1H, H5′); 6.82-6.58 (m, 4H, H6, H6′, H8, H8′); 5.27 (d, J=7.2 Hz, 1H, H5-furan); 3.35 (d, J=7.2 Hz, 1H, H4furan); 3.18 (s, 3H, CH₃). ¹³C-NMR (300 MHz, DMSO-d₆): δ/ppm: 168.52 (C4); 167.20 (C4′); 164.39 (C2); 160.58 (C2′); 159.38 (C7′); 159.22 (C7); 154.32 (C9′); 154.16 (C9); 125.42 (C5); 125.11 (C5′); 111.81 (C6); 111.55 (C6′); 106.55 (C8′); 106.20 (C8); 101.34 (C10); 96.53 (C10′); 96.38 (C3′); 96.19 (C3); 94.75 (C5-furan); 44.24 (CH₃); 28.15 (C4-furan).

### EXAMPLE 4

### Bis-(4,7-dihydroxy-2-oxo-2H-chromen-3-yl)-acetaldehyde

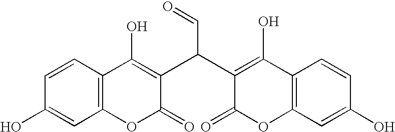

Bis-(4,7-dihydroxy-2-oxo-2H-chromen-3-yl)-acetaldehyde was separated as the second fraction in the reaction disclosed in Example 3. 60.0 mg (10%) of the product were obtained. M.p.:>300° C.

Elemental analysis: C₂₀H₁₂O₉ (Mᵣ=396.31) Calc. : C=60.61% H=3.05%. Found: C=60.33% H=3.42%. MS m/z EI: 395.3 (M−H). IR(KBr): v/cm⁻¹: 3341; 1691; 1664; 1584; 1428; 1294; 767. ¹H-NMR (300 MHz, DMSO-d₆): δ/ppm: 10.78 (bs, 2H, C4-OH); 10.60 (bs, 2H, C7-OH); 7.88 (d, J=8.4 Hz, 2H, H5); 7.77 (d, J=8.4 Hz, 2H, H6); 7.12 (s, 2H, H8); 6.74 (s, 1H, CH). ¹³C-NMR (300 MHz, DMSO-d₆): δ/ppm: 172.48 (CHO); 166.62 (C4); 165.94 (C4′); 162.89 (C2); 162.31 (C2′); 161.82 (C7′); 160.99 (C7); 160.79 (C9′); 159.07 (C9); 157.99 (C5); 157.53 (C5′); 154.92 (C6); 154.49 (C6′); 126.04 (C8′); 125.29 (C8); 113.94 (C10); 113.83 (C10′); 108.69 (C3′); 108.17 (C3); 92.67 (CH).

### EXAMPLE 5

### 7,8-Dihydroxy-3-(4,6,7-trihydroxy-2-oxo-2H-chromen-3-yl)-furo[3,2-c]chromen-4-one

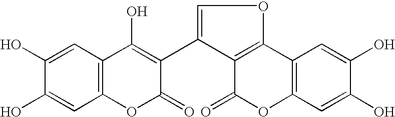

A 30% aqueous glyoxal solution (0.25 ml; 1.42 mmol) was added to a solution of 4,6,7-trihydroxycoumarin (1.00 g; 5.15 mmol) in ethanol (5.0 ml) and heated at the temperature of the boiling point for 36 hours. The reaction mixture was evaporated to half its volume with a greenish precipitate of 7,8-dihydroxy-3-(4,6,7-trihydroxy-2-oxo-2H-chromen-3-yl)-furo[3,2-c]chromen-4-one (0.18 g; 12%) separating from the solution. M. p.>300° C.

Elemental analysis: C₂₀H₁₀O₁₀ (Mᵣ=410.29) Calc.: C=58.55% H=2.46%. Found: C=57.98% H=2.40%. MS m/z EI: 409.3 (M−H). IR(KBr): v/cm⁻¹: 3361; 1697; 1666; 1581; 1437; 1293; 765. ¹H-NMR (300 MHz, DMSO-d₆): δ/ppm: 10.48 (bs, 1H, C4-OH); 10.21 (bs, 2H, C7-OH); 9.66 (bs, 2H, C6-OH); 7.34 (s, 1H, H5); 7.19 (s, 1H, H5′); 7.10 (s, 1H, H5-furan); 6.92 (s, 1H, H8); 6.78 (s, 1H, H8′); 3.45 (s, 1H, H3-furan). ¹³C-NMR (300 MHz, DMSO-d₆): δ/ppm: 164.16 (C4-OH); 161.24 (C4′); 158.30 (C2); 157.52 (C2′); 152.21 (C7′); 149.86 (C7); 148.63 (C9′); 148.06 (C9); 147.18 (C6); 143.86 (C6′); 143.45 (C10); 143.43 (C1O′); 108.69 (C5-furan); 108.59 (C3); 108.42 (C5); 107.39 (C3′); 105.25 (C5′); 103.97 (C8); 102.83 (C8′); 93.04 (C4-furan).

### EXAMPLE 6

### 6,7-Dihydroxy-2-methoxy-3-(4,7,8-trihydroxy-2-oxo-2H-chromen-3-yl)-2,3-dihydro-furo[3,2-c]chromen-4-one

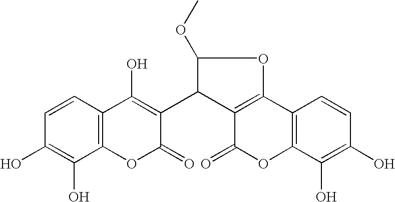

A 30% aqueous glyoxal solution (0.13 ml; 0.74 nrnol) was added to a solution of 4,7,8-trihydroxycoumarin (0.50 g; 2.58 mmol) in methanol (5.0 ml) and stirred at room temperature for 240 hours with a brown precipitate separating from an orange solution. The reaction mixture was left overnight at 4° C. and filtered. 6,7-Dihydroxy-2-methoxy-3-(4,7,8-trihydroxy-2-oxo-2H-chromen-3-yl)-2,3-dihydro-furo[3,2-c]-chromen-4-one (0.08 g) was obtained.

Elemental analysis: C₂₁H₁₄O₁₁ (Mᵣ=442.23) Calc.: C=57.02% H=3.19%. Found: C=57.48% H=2.82%. MS m/z EI: 441.0 (M−H). IR (KBr): v/cm⁻¹: 3343; 1694; 1661; 1584; 1296; 768. ¹H-NMR (300 MHz, DMSO-d₆): δ/ppm: 10.48 (bs, 1H, C4-OH); 10.21 (bs, 2H, C7-OH); 9.66 (bs, 2H, C6-OH); 7.34 (s, 1H, H5); 7.19 (s, 1H, H5′); 7.10 (s, 1H, H5-furan); 6.92 (s, 1H, H8); 6.78 (s, 1H, H8′); 3.45 (s, 1H, H4-furan); 3.24 (s, 3H, CH₃).

### EXAMPLE 7

### 3,3′,3″3′″-(1,4-Propane)tetrakis[4-hydroxycoumarin]

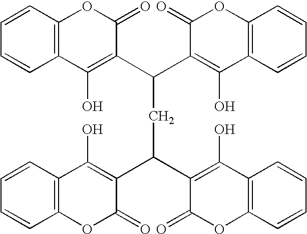

Malonaldehyde (0.14 ml; 0.85 mmol) was added to a solution of 4-hydroxycoumarin (0.50 g; 3.08 mmol) in 96% ethanol (10.0 ml). The reaction mixture was heated at the boiling temperature for six hours and an abundant white precipitate was formed. The obtained precipitate was filtered in vacuo and then boiled in absolute ethanol. The obtained 3,3′,3″3′″-(1,4-propane)tetrakis[4-hydroxycoumarin] was filtered and dried (0.44 g; 83%). M.p. 286-288° C.

Elemental analysis: C₃₉H₂₄O₁₂ (Mᵣ=684.60) Calc.: C=68.42% H=3.53%. Found: C=68.11% H=3.67%. MS m/z ES: 683.3 (M−H). IR(KBr): v/cm⁻¹: 3368; 1645; 1612; 1560; 1298; 760. ¹H-NMR (300 MHz, DMSO-d₆): δ/ppm: 10.16 (bs, 4H, C4-OH); 7.95 (d, J=7.8 Hz, 4H, H5); 7.61 (t, J=7.8 Hz, 4H, H7); 7.40-7.35 (m, 8H, H6 and H8), 7.12 (2 d, ArH). ¹³C-NMR (300 MHz, DMSO-d₆): δ/ppm: 165.34 (C4); 164.74 (C2); 152.41 (C9); 136.76 (ArC1′); 132.54 (C7); 127.04 (ArC2′); 124.33 (C6); 124.13 (C5); 117.46 (C10); 116.38 (C8); 104.63 (C3); 37.77 (CH).

### EXAMPLE 8

### 4-(4,7-Dihydroxy-2-oxo-2H-chromen-3-yl)-2H-pyrano[2,3-b]chromen-5-one

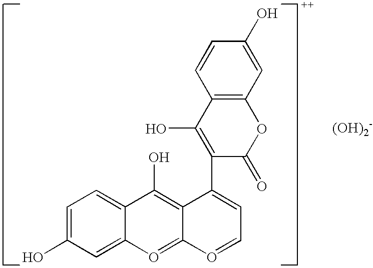

Malonaldehyde (0.52 ml; 3.08 mmol) was added to a solution of 4,7-dihydroxycoumarin (2.00 g; 11.23 mmol) in methanol (40.0 ml). The reaction mixture was stirred at room temperature for 60 hours during which the reaction mixture took on an intensive reddish-purple colour. By evaporating the reaction mixture a pink precipitate was obtained and it was filtered it? vacuo. By standing in the air the precipitate of 4-(4,7-dihydroxy-2-oxo-2H-chromen-3-yl)-2H-pyrano[2,3-b]chromen-5-one became sticky and by dissolution in methanol the solution took on an intensive colour again. Only a minor part of the compound could be isolated (0.02 g). M. p.:>300° C.

Elemental analysis: C₂₂H]₅O₈ (Mᵣ=407.35) Calc. : C=64.87% H=3.71%. MS t/z ES: 406 (M−H). IR(KBr): v/cm⁻¹: 3364; 1641; 1624; 1558; 1294; 767. ¹H-NMR (300 MHz, DMSO-d₆): δ/ppm: 12.13 (bs, 1H, C4-OH); 10.14 (bs, 2H, C7-OH); 7.62-6.91 (m, 8H, ArH).

### EXAMPLE 9

### 4-(4,5,7-Trihydroxy-2-oxo-2H-chromen-3-yl)-2H-pyrano[2,3-b]chromen-5-one

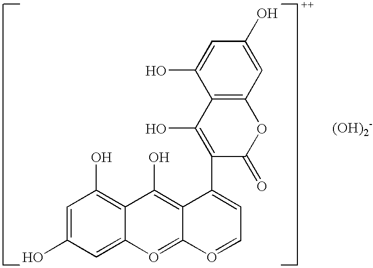

Malonaldelhyde (0.24 ml; 1.42 mmol) was added to a solution of 4,5,7-trihydroxycoumarin (1.00 g; 5.16 mmol) in 96% ethanol (20.0 ml). The reaction mixture was heated at the boiling temperature of ethanol for five hours during which the reaction mixture took on an intensive reddish-pink colour. By evaporating the reaction mixture a pink precipitate was obtained and it was filtered in vacuo. 4-(4,5,7-Trihydroxy-2-oxo-2H-chromen-3-yl)-2H-pyrano[2,3-b]chromen-5-one (0.32 g; 43%) was obtained. M. p.: 254-256° C.

Elemental analysis: [C₂₁H₁₂O₁₀]⁺⁺(OH)₂⁻⁻ (Mᵣ=423.298) Calc.: C=55.03% H=3.08%. Found: C=55.35% H=3.24%. MS nilz ES: 422.9 (M−H). IR (KBr): v/cm⁻¹: 3364; 1641; 1624; 1558; 1294; 767. ¹H-NMR (300 MHz, DMSO-d₆): δ/ppm: 12.01 (bs, 1H, C4-OH); 10.11 (bs, 2H, C7-OH); 7.62 (bs, 2H, C5-OH); 8.53-8.21 (m, 2H, ArH); 8.13-7.92 (m, 2H, ArH); 6.01-5.93 (m, 2H, ArH).

### EXAMPLE 10

### 4-(4,6,7-Trihydroxy-2-oxo-2H-chromen-3-yl)-2H-pyrano[2,3-b]chromen-5-one

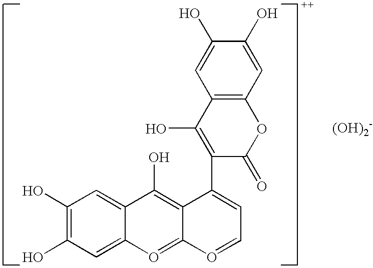

Malonaldehyde (0.24 ml; 1.42 mmol) was added to a solution of 4,6,7-trihydroxycoumarin (1.00 g; 5.16 mmol) in 96% ethanol (20.0 ml). The reaction mixture was heated at the boiling temperature of ethanol for 32.5 hours. By evaporating the reaction mixture a sandy-coloured precipitate was obtained and it was filtered in vacuo. 4-(4,6,7-Trihydroxy-2-oxo-2H-chromen-3-yl)-2H-pyrano[2,3-b]chromen-5-one (0.24 g; 41%) was obtained. M. p.: 258-260° C.

Elemental analysis: [C₂₁H₂O₁₀]⁺⁺(OH)₂⁻⁻ (Mᵣ=423.298) Calc.: C=55.03% H=3.08%. Found: C=55.41% H=3.23%. MS m/z ES: 423 (M−H). IR (KBr): v/cm⁻¹: 3361; 1647; 1624; 1558; 1290; 765. ¹H-NMR (300 MHz, DMSO-d₆): δ/ppm: 12.21 (bs, 1H, C4-OH); 10.13 (bs, 2H, C7-OH); 8.83 (bs, 2H, C6-OH); 8.70-7.94 (m, 4H, ArH); 6.00-5.77 (m, 2H, ArH).

### EXAMPLE 11

### 3,3′,3 ″3′″-(1,5-Pentane)tetrakis[4-hydroxycoumarin]

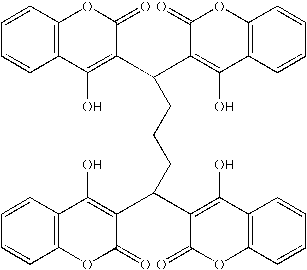

Glutardialdehyde (0.14 ml; 0.77 mmol) was added to a solution of 4-hydroxycoumarin (1.00 g; 6.17 mmol) in ethanol (10.0 ml) and heated at the boiling temperature of ethanol for nine hours. 3,3′,3″,3′″-(1,5-Pentane)tetrakis[4-hydroxycoumarin] (0.67 g; 63%) with m.p. 291-294° C. was obtained.

Elemental analysis: C₄₁H₂₈O₁₂ (Mᵣ=712.67) Calc. : C=69.10% H=3.96%. Found: C=69.14% H=3.81%. MS mn/z EI: 711.5 (M−H). IR(KBr): v/cm⁻¹: 3361; 1697; 1666; 1581; 1437; 1293; 765. ¹H-NMR (300 MHz, DMSO-d₆): δ/ppm: 9.57 (bs, 4H, C4-OH); 7.95 (d, J=7.8 Hz, 4H, H5); 7.61 (t, J=7.8 Hz, 4H, H7); 7.40-7.35 (m, 8H, H6 and H8); 4.59 (t, 2H, H2 and H4-pentane); 3.57 (m, 1H, H3-pentane). ¹³C-NMR (300 MHz, DMSO-d₆): δ/ppm: 165.26 (C4); 163.55 (C2); 151.83 (C9); 132.17 (C7); 124.20 (C5); 123.62 (C6); 116.90 (C10); 116.14 (C8); 105.48 (C3); 37.77 (CH); 32.34 (C2-pentane); 32.14 (C4-pentane); 27.18 (C3-pentane).

### EXAMPLE 12

### 3,3′,3″,3′″-(1,5-Pentane)tetrakis[4,7-dihydroxycoumarin]

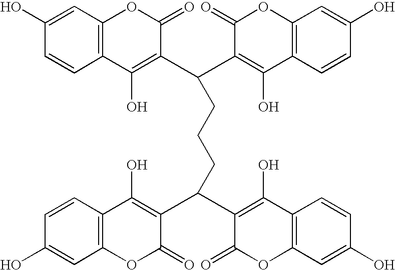

Glutardialdehyde (0.14 ml; 0.77 mmol) was added to a solution of 4,7-dihydroxycoumarin (0.50 g; 2.81 mmol) in methanol (10.0 ml) and stirred at room temperature for 14 hours. 3,3′,3″3′″-(1,5-Pentane)tetrakis[4,7-dihydroxycoumarin] (0.27 g; 25%) of m.p. 299-302° C. was obtained.

Elemental analysis: C₄₁H₂₈O₁₆ (Mᵣ=776.65) Calc. : C=63.41% H=3.63%. Found: C=63.12% H=3.81%. MS m/z EI: 775 (M−H). IR(KBr): v/cm⁻¹: 3344; 1694; 1662; 1581; 1297; 769. ¹H-NMR (300 MHz, DMSO-d₆): δ/ppm: 12.30 (bs, 41H, C4-OH); 10.21 (bs, 4H, C7-OH); 7.72 (d, J=8.4 Hz, 4H, H5); 7.51 (d, J=8.4 Hz, 4H, H6); 7.14 (s, 4H, H8); 3.56 (t, J=7.8 Hz, 2H, CH); 1.47 (m, 4H, CH₂); 1.32 (h, 2H, CH₂). ¹³C-NMR (300 MHz, DMSO-d₆): δ/ppm: 164.41 (C4); 162.18 (C2); 156.91 (C7); 152.25 (C9); 149.18 (C5); 123.12 (C6); 108.47 (C8); 98.51 (C3); 32.14 (CH₂); 26.71 (CH₂); 19.81 (CH).

### EXAMPLE 13

### 3,3′,3″,3′″-(1,5-Pentane)tetrakis[4,5,7-trihydroxycoumarin]

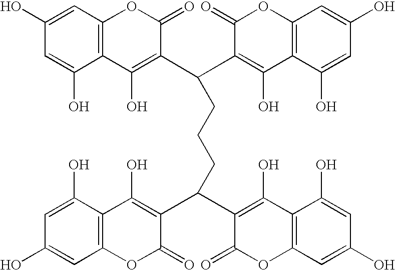

Glutardialdehyde (0.12 ml; 0.71 mmol) was added to a solution of 4,5,7-trihydroxycoumarin (0.50 g; 2.58 mmol) in methanol (5.0 ml) and stirred at room temperature for 12 hours. 3,3′,3″3′″-(1,5-Pentane)tetrakis[4,5,7-trihydroxycoumarin] (0.19 g; 36%) of m.p.>300° C. was obtained.

Elemental analysis: C₄₁H₂₈O₂₀ (Mᵣ=840.65) Calc. : C=58.58% H=3.36%. Found: C=58.21% H=3.94%. MS m/z EI: 839 (M−H). IR (KBr): v/cm⁻¹: 3330; 1664; 1582; 1441; 1294; 767. ¹H-NMR (300 MHz, DMSO-d₆): δ/ppm: 12.21 (bs, 4H, C4-OH); 10.30 (bs, 4H, C7-OH); 6.93 (bs, 4H, C5-OH), 6.31-5.98 (m, 8H, H6 and H8); 3.57 (m, 2H, CH); 1.47 (m, 6H, CH₂ and CH₂). ¹³C-NMR (300 MHz, DMSO-d₆): δ/ppm: 164.40 (C4); 162.12 (C2); 156.87 (C9); 155.23 (C6); 150.32 (C7); 121.11 (C10); 103.19 (C8); 102.17 (C6); 98.90 (C3); 32.22 (CH₂); 26.41 (CH1); 17.33 (CH).

### EXAMPLE 14

### 3,3′,3″,3′″-(1,5-Pentane)tetrakis[4,6,7-trihydroxycoumarin]

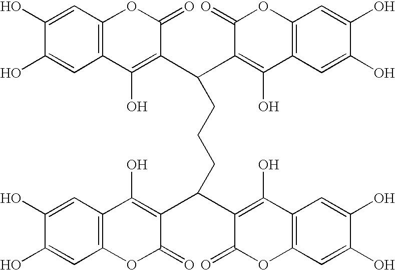

Glutardialdehyde (0.12 ml; 0.71 mmol) was added to a solution of 4,6,7-trihydroxycoumarin (0.50 g; 2.58 mmol) in methanol (5.0 ml) and heated at room temperature for 8.5 hours. 3,3′,3″,3′″-(1,5-Pentane)tetrakis[4,6,7-trihydroxy-coumarin] (0.14 g; 26%) of m.p.>300° C. was obtained.

Elemental analysis: C₄₁H₂₈O₂₀ (Mᵣ=840.65) Calc. : C=58.58% H=3.36%. Found: C=58.31% H=3.47%. MS m/z EI: 839 (M−H). IR(KBr): v/cm⁻¹: 3341; 1687; 1664; 1584; 1290; 765. ¹H-NMR (300 MHz, DMSO-d₆): δ/ppm: 12.04 (bs, 4H, C4-OH); 10.12 (bs, 4H, C7-OH); 9.43 (bs, 4H, C6-OH), 7.21 (s, 4H, H5); 6.31 (s, 4H, H8); 3.12 (m, 2H, CH); 1.51-1.18 (m, 6H, CH₂ and CH₂). ¹³C-NMR (300 MHz, DMSO-d₆): δ/ppm: 164.23 (C4); 162.09 (C2); 156.11 (C7); 155.81 (C6); 153.18 (C9); 123.12 (C10); 115.12 (C5); 108.35 (C8); 88.72 (C3); 32.14 (CH₂); 29.19 (CH₂); 15.41 (CH).

### EXAMPLE 15

### 3,3′,3″,3′″-(1,5-Pentane)tetrakis[4,7,8-trihydroxycoumarin]

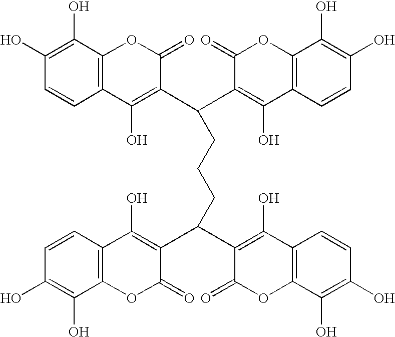

Glutardialdehyde (0.12 ml; 0.71 mmol) was added to a solution of 4,7,8-trihydroxy-coumarin (0.50 g; 2.58 mmol) in methanol (5.0 ml) and stirred at room temperature for 16 hours. 3,3′,3″3′″-(1,5-Pentane)tetrakis[4,7,8-trihydroxycoumarin] (0.14 g; 26%) of m.p. >300° C. was obtained.

Elemental analysis: C₄₁H₂₈O₂₀ (Mᵣ=840.65) Calc. : C=58.58% H=3.36%. Found: C=58.64% H=3.25%. MS m/z EI: 839 (M−H). IR (KBr): v/cm⁻¹: 3321; 1694; 1647; 1582; 1294; 772. ¹H-NMR (300 MHz, DMSO-d₆): δ/ppm: 12.04 (bs, 4H, C4-OH); 10.07-9.51 (bs, 8H, C7- and C8-OH); 7.27 (d, J=8.4 Hz, 4H, H5); 6.67 (d, J=8.4 Hz, 4H, H6); 3.07 (t, 2H, CH); 1.59-1.37 (m, 6H, CH₂ and CH₂). ¹³C-NMR (300 MHz, DMSO-d₆): δ/ppm: 164.41 (C4); 162.11 (C2); 151.17 (C7); 142.41 (C9); 139.15 (C8); 125.11 (C10); 118.31 (C5); 121.61 (C6); 89.15 (C3); 30.21 (CH₂); 28.87 (CH₂); 14.71 (CH).

### EXAMPLE 16

### 2-[Bis-(4,7-dihydroxy-2-oxo-2H-chromen-3-yl)-methyl]-benzaldehyde

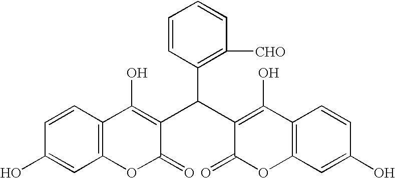

Phthaldialdehyde (0.36 g; 2.56 mmol) was added to a solution of 4,7-dihydroxycoumarin (2.00 g; 10.3 mmol) in dry acetone (90.0 ml). The reaction mixture was heated at the boiling point for 50 hours. After the completion of the reaction the solvent was evaporated to one third of the starting volume and the evaporation residue was left overnight at 13° C. After filtration in vacuo, a shiny light yellow precipitate remained. The obtained product 2-[bis-(4,7-dihydroxy-2-oxo-2H-chromen-3-yl)-methyl]-benzaldehyde was recrystallized from ethanol (0.80 g; 33%). M. p. >300° C.

Elemental analysis: C₂₆H₁₆O₉ (Mᵣ=472.388) Calc. : C=66.10% H=3.41%. Found: C=66.17% H=3.85%. MS m/z ES-: 471 (M−H). IR(KBr): v/cm⁻¹: 3445; 1660; 1617; 1579; 1370; 1294; 761. ¹H-NMR (300 MHz, DMSO-d₆): δ/ppm: 12.25 (bs, 2H, C4-OH); 10.86 (bs, 1H, CHO); 8.29 (s, 2H, C7-OH); 8.14 (d, J=8.7 Hz, 1H, ArH6); 7.80 (t, J=7.8 Hz, 2H, H5); 7.49 (t, J=6.6 Hz, 1H, ArH4); 7.38-7.30 (m, 2H, ArH3, ArH5); 6.95 (d, 2H, J=8.7 Hz, H6); 6.76 (s, 2H, H8); 5.82 (s, 1H, CH). ¹³C-NMR (300 MHz, DMSO-d₆): δ/ppm: 191.67 (CHO); 162.67 (C4); 160.64 (C2); 154.45 (C7); 154.24 (C9); 145.40 (ArC1); 134.57 (C5); 134.24 (ArC2); 129.96 (C6); 127.55 (ArC3); 126.75 (ArC6); 124.79 (ArC4); 113.98 (ArC5); 104.77 (C10); 102.54 (C8), 101.02 (C3); 27.67 (CH).

### EXAMPLE 17

### 2-[Bis-(4,7,8-trihydroxy-2-oxo-2H-chromen-3-yl)-methyl]-benzaldehyde

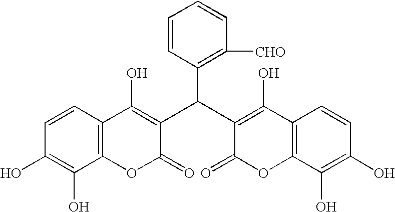

Phthaldialdehyde (0.10 g; 0.75 mmol) was added to a solution of 4,7,8-trihydroxycoumarin (0.50 g; 2.58 mmol) in 96% ethanol (10.0 ml). The reaction mixture was heated at the boiling point for 38 hours. The reaction mixture was left overnight at 4° C. and a dark purplish-brown precipitate was obtained. The obtained 2-[bis-(4,7,8-trihydroxy-2-oxo-2H-chromen-3-yl)-methyl]-benzaldehyde was recrystallized from glacial acetic acid (0.28 g; 43%). M. p. >300° C.

Elemental analysis: C₂₆H16O₁₁ (Mᵣ=504.388) Calc. : C=63.30% H=2.97%. Found: C=62.94% H=3.09%. MS m/z ES−: 503 (M−H). IR (KBr): v/cm⁻¹: 3445; 1737; 1673; 1652; 1593; 1320; 1262; 792. ¹H-NMR (300 MHz, DMSO-d₆): δ/ppm: 12.40 (bs, 2H, C4-OH); 11.90 (bs, 1H, CHO); 10.07 (bs, 2H, C7-OH), 9.05 (bs, 2H, C8-OH); 8.36 (d, J=8.7 Hz, 1H, 2H, H5); 7.99 (d, J=7.8 Hz, 1H, ArH5); 7.72 (m, 3H, H6, ArH3); 7.62 (m, 2H, ArH4, ArH6); 3.63 (s, 1H, CH). ¹³C-NMR (300 MHz, DMSO-d₆): δ/ppm: 195.45 (CHO); 165.85 (C4); 160.79 (C2); 152.66 (C7); 149.55 (ArC1); 146.55 (C9); 142.11 (ArC4); 138.40 (C8); 134.62 (ArC4); 133.44 (ArC2); 133.10 (ArC6); 132.94 (C5); 131.05 (ArC3); 130.90 (ArC5); 129.21 (C10); 128.78 (C6); 123.15 (C3); 21.17 (CH).

### EXAMPLE 18

### 7-(4-Hydroxy-2-oxo-2H-chromen-3-yl)-7H-chromeno[4,3-b]-chromen-6-one

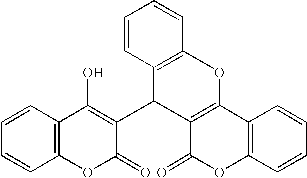

Phtlhaldialdehyde (0.41 g; 2.56 mmol) was added to a solution of 4-hydroxycoumarin (2.00 g; 12.4 mmol) in 96% ethanol (15.0 ml). The reaction mixture was heated at the boiling point for 39 hours. By standing overnight at 13° C. a yellow precipitate was obtained. The obtained precipitate of 7-(4-hydroxy-2-oxo-2H-chromen-3-yl)-7H-chromeno[4,3-b]-chromen-6-one was filtered in vacuo and then recrystallized from a 50% aqueous N,N-dimethyl acetamnide solution. The obtained precipitate was filtered in vacuo and dried (0.99 g; 39%). M. p.>255° C. (dec.).

Elemental analysis: C₂₅H₁₄O₈ (Mᵣ=442.362) Calc. : C=67.88% H=3.19%. Found: C=67.51% H=3.61%. MS m/z ES−: 441 (M−H). IR (KBr): v/cm⁻¹: 3411; 1662; 1614; 1340; 787. ¹H-NMR (300 MHz, DMSO-d₆): δ/ppm: 12.58 (bs, 1H, C4-OH); 8.19 (d, J=7.8 Hz, 1H, H5); 8.15 (d, J 7.5 Hz, 1H, H5′), 7.83 (t, J=6.6 Hz, 1H, H7); 7.75 (t, J=6.6 Hz, 1H, H7′); 7.68-7.29 (m, 6H, H8, H8′, ArH); 7.23 (m, 2H, H6 and H6′); 4.70 (s, 1H, CH). ³C-NMR (300 MHz, DMSO-d₆): δ/ppm: 167.88 (C4); 165.99 (C4′); 162.45 (C2); 162.31 (C2′); 156.83 (ArC2); 152.41 (C9); 152.06 (C9′); 139.66 (ArC3); 132.97 (C7); 132.82 (C7′); 128.86 (C10); 128.13 (C1O′); 126.61 (ArC5); 124.79 (C5); 124.53 (C5′); 124.33 (C6); 124.18 (C6′); 123.45 (ArC4); 123.22 (ArC2); 116.61 (C8); 116.29 (C8′); 115.44 (ArC6); 91.19 (C3, C3′); 26.41 (CH).

### EXAMPLE 19

### 1,3-Dihydroxy-7-(4,5,7-trihydroxy-2-oxo-2H-chromen-3-yl)-7H-chromeno[4,3-b]-chromen-6-one

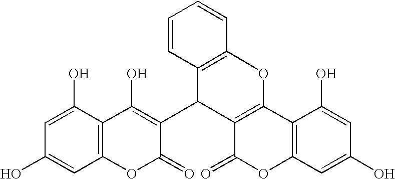

Phthaldialdehyde (0.38 g; 5.67 mmol) was added to a solution of 4,5,7-trihydroxycoumarin (2.00 g; 10.3 mmol) in dry acetone (60.0 ml), it was heated at the boiling point of acetone for 109 hours and an abundant orange precipitate was formed. Into the reaction flask dichloromethane (25 ml) was added under stirring for two hours at room temperature. After filtration in vacuo and drying, the precipitate of 1,3-dihydroxy-7-(4,5,7-trihydroxy-2-oxo-2H-chromen-3-yl)-7H-chromeno[4,3-b]-chromen-6-one of a yellowish orange colour was obtained (1.58 g; 65%). The obtained product was recrystallized from a 50% aqueous acetic acid solution. M. p.:>300° C.

Elemental analysis: C₂₅H₁₄O₁₀ (Mᵣ=474.362) Calc. : C=63.30% H=2.97%. Found: C=63.19% H=3.20%. MS m/z ES−: 473 (M−H). IR(KBr): v/cm⁻¹: 3417; 1664; 1633; 1584; 1349; 782. ¹H-NMR (300 MHz, DMSO-d₆): δ/ppm: 12.25 (bs, 2H, C4-OH); 10.15 (bs, 2H, C7-OH); 9.50 (bs, 2H, C6-OH); 7.11 (s, 2H, H5); 6.89-6.70 (m4H, ArH); 6.62 (s, 2H, H8); 5.39 (s, 1H, CH). ¹³C-NMR (300 MHz, DMSO-d₆): δ/ppm: 166.64 (C4); 163.38 (C4′); 162.11 (C2); 157.41 (ArC1); 151.23 (C9); 148.87 (C7); 142.99 (C6); 129.91 (ArC3); 128.15 (ArC2); 128.03 (ArC5); 122.11 (ArC4); 120.51 (C10); 107.59 (C8); 107.40 (ArC6); 103.02 (C3); 88.46 (C3′); 33.18 (CH).

### EXAMPLE 20

### 1,3-Dihydroxy-7-(4,6,7-trihydroxy-2-oxo-2H-chromen-3-yl)-7H-chromeno[4,3-b]-chromen-6-one

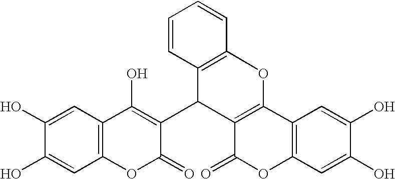

Phthaldialdehyde (0.38 g; 5.67 mmol) was added to a solution of 4,6,7-trihydroxycoumarin (2.00 g; 10.3 mmol) in dry acetone (150.0 ml) and it was heated at the boiling point of acetone for 90 hours. After the completion of the reaction the solvent was evaporated to one third of the starting volume and the evaporation residue was left overnight at 13° C. After filtration in vacuo a yellowish brown precipitate remained. The obtained product 1,3-dihydroxy-7-(4,6,7-trihydroxy-2-oxo-2H-chromen-3-yl)-7H-chromeno[4,3-b]-chromen-6-one was recrystallized from ethanol (0.62 g; 25%). M. p.:>300° C.

Elemental analysis: C₂₅H14O₁₀ (Mᵣ=474.362) Calc. : C=63.30% H=2.97%. Found: C=63.12% H=3.42%. MS m/z ES−: 473.4 (M−H). IR(KBr): v/cm⁻¹: 3425; 1645; 1570; 1238; 818. ¹H-NMR (300 MHz, DMSO-d₆): δ/ppm: 12.25 (bs, 2H, C4-OH); 10.15 (bs, 2H, C7-OH), 9.50 (bs, 2H, C6-OH); 7.11 (s, 2H, H5); 6.89-6.70 (m4H, ArH); 6.62 (s, 2H, H8); 5.39 (s, 1H, CH). ¹³C-NMR (300 MHz, DMSO-d₆): δ/ppm: 166.64 (C4); 163.38 (C4′); 162.11 (C2); 157.41 (ArC1); 151.23 (C9); 148.87 (C7); 142.99 (C6); 129.91 (ArC3); 128.15 (ArC2); 128.03 (ArC5); 122.11 (ArC4); 120.51 (C10); 107.59 (C8); 107.40 (ArC6); 103.02 (C3); 88.46 (C3′); 33.18 (CH).

1. Products of condensation of hydroxycoumarin derivatives with aromatic and aliphatic dialdehydes of the general formulae I, II, and IV 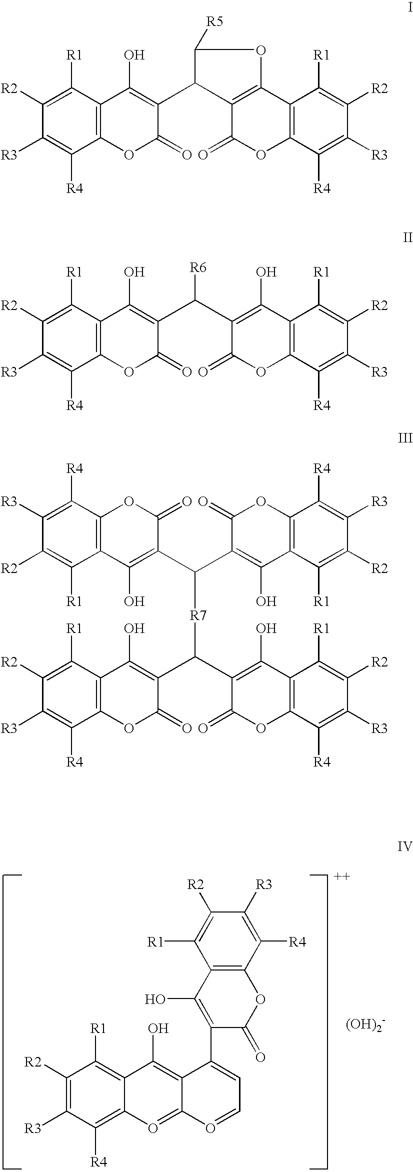 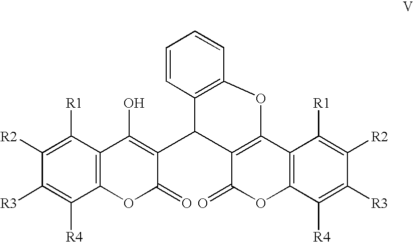
wherein
is a single or a double bond;
R1=R2=R3=R4=H or
R1=R2=R4=H, R3=OR or
R2=R4=K R1=R3=OH or
R1=R4=H, R2=R3=OH or
R1=R2=H, R3=R4=OH;
R5=H, OCH₃ or OCH₂CH₃;
R6=o-C₆H₄—CHO, CHO;
R7=(CH₂)ₙ and n=1-3;
and pharmaceutically acceptable salts and esters thereof

2. Compound of the general formula I according to claim 1, characterized in that—is a single bond, R1=R2=R3=R4=H, R5=OCH₂CH₃.

3. Compound of the general formula I according to claim 1, characterized in that—is a double bond, R1=R2=R4=H, R3=OH.

4. Compound of the general formula I according to claim 1, characterized in that—is a single bond, R1=R2=R4=H, R3=OH, R5=OCH₃.

5. Compound of the general formula II according to claim 1, characterized in that R1=R2=R4=H, R3=OH, R6=CHO.

6. Compound of the general formula I according to claim 1, characterized in that—is a double bond, R1=R4=H, R2=R3=OH, R6=H.

7. Compound of the general formula I according to claim 1, characterized in that—is a single bond, R1=R2=H, R3=R4=OH, R5=OCH₃.

8. Compound of the general formula III according to claim 1, characterized in that R1=R2=R3=R4=H, R7=CH₂.

9. Compound of the general formula IV according to claim 1, characterized in that R1=R2=R4=H, R3=OH.

10. Compound of the general formula IV according to claim 1, characterized in that R2=R4=H, R1=R3=OH.

11. Compound of the general formula IV according to claim 1, characterized in that R1=R4=H, R2=R3=OH.

12. Compound of the general formula III according to claim 1, characterized in that R1=R2=R3=R4=H, R7=(CH₂)ₙ, n=3.

13. Compound of the general formula III according to claim 1, characterized in that R1=R2=R4=R3=OH, R7=(CH₂)ₙ, n=3.

14. Compound of the general formula III according to claim 1, characterized in that R=R4=H, R₁=R3=OH, R7=(CH₂)ₙ, n=3.

15. Compound of the general formula III according to claim 1, characterized in that R1=R4=H, R2=R3=OH, R7=(CH₂)ₙ, n=3.

16. Compound of the general formula III according to claim 1, characterized in that R1=R2=H, R3=R4=OH, R7=(CH₂)ₙ, n=3.

17. Compound of the general formula II according to claim 1, characterized in that R1=R2=R4=H, R3=OH, R6=o-C₆H₄—CHO.

18. Compound of the general formula II according to claim 1, characterized in that R1=R2=H, R3=R4=OH, R6=o-C6H4—CHO.

19-21. (canceled)

22. Process for the preparation of compounds of the general formulae I-IV and pharmaceutically acceptable salts and esters thereof, wherein—is a single or a double bond; R1=R3=R4=H or R1=R2=R4=H R3=OH or R2=R4=H, R1=R3=OH or R1=R4=H, R=R3=OH or R1=R2=H, R3=R4=OH; R5=H, OCH₃ or OCH₂CH₃; R6=o-C₆H₄—CHO, CHO; R7=(CH₂)ₙ and n=1-3, characterized in that hydroxycoumarins of the general formula VI, wherein R1=R2=R3=R4=H or R1=R2=R4H, R3=OH or R2=R4H, R1=R3=OH or R1=R4=H, R2=R3=OH or R1=R2=H, R3=R4=OH, are subjected to a reaction with dialdehydes of the general formula VII, wherein R8 has the meaning of ortho-phenyl or (CH₂)ₙ and n=0-3, in a suitable solvent at a temperature from room temperature to the boiling temperature of the solvent to give compounds of the general formulae I-IV, which are optionally subjected to separation on a silica gel column using the solvent system CH₂Cl₂:CH₃OH(5:1).

23. Process according to claim 22, characterized in that the suitable organic solvent is methanol, ethanol or acetone.

24. Pharmaceutical formulation suitable for treating viral infections in humans, containing a virostatically effective amount of the compounds of the general formulae I-IV or pharmaceutically acceptable salts and esters thereof according to claim 1 in combination with pharmaceutically acceptable carriers.

25. (canceled).

26. Compounds of the general formulae I, II, III, IV for use in the treatment of HIV infection.

27. Use of compounds of the general formula I, II, III, IV for the manufacture of a medicament for the treatment of HIV infection.
